(19) United States
(12) Patent Application Publication
(10) Pub. No.: US 2007/0197652 A1
(43) Pub. Date: Aug. 23, 2007

(54) SOLID PHARMACEUTICAL COMPOSITION FOR THE TREATMENT OF THE BENIGN PROSTATIC HYPERPLASIA ITS PREPARATION PROCEDURE AND ITS THERAPEUTICAL METHOD

(76) Inventor: Hipolito Carmelo Maria Barreiro, Buenos Aires (AR)

Correspondence Address: Sanchelima and Associates, P. A.;Jesus Sanchelima, Esq., 235 S.W. Le Jeune Rd., Miami, FL, 33134, US

(21) Appl. No.: 11/676,277

(22) Filed: Feb. 17, 2007

(30) Foreign Application Priority Data
Feb. 17, 2006 (AR) P20060100573

(51) Int. Cl. A61K 31/17

(52) U.S. Cl. 514/586

(57) ABSTRACT

We are hereby dealing with a solid pharmaceutical composition for the treatment of hyperplasia benign of prostate, also of a manufacturing procedure and the treatment method, where the pharmaceutical composition at least includes between 0.5 and 5 mg of a polysaccharide extract from a Gram negative bacteria wall and also pharmaceutically acceptable excipients. The aforesaid bacteria are preferably Pseudomonas Aeruginosa. Excipients can be i.e. colloidal silicic anhydride, wheat starch, micro crystalline cellulose, lactose or a combination of same. The composition may be in form of pastilles, capsules, tablets or pills.

### BACKGROUND OF THE INVENTION

1. Field of the Invention

The present invention refers to a solid pharmaceutical composition for the treatment of the Benign Prostate Hyperplasia (BPH), its preparation procedure and therapeutic method, in which the pharmaceutical composition contains at least, between 0.5 and 5 mg. of a Polysaccharide extract from the wall of gram negative bacteria and a pharmaceutically acceptable excipient. Said bacteria mainly are Pseudomonas Aeruginosa. The excipient may be e.g.: salicylic colloidal anhydrid, wheat starch, micro crystalline cellulose, lactose or a combination of both. The composition may be produced as pills, capsules, tablets or pellets.

2. Description of the Related Art

Benign Prostatic Hyperplasia

Benign Prostate Hyperplasia (BPH) is a common age related proliferative abnormality of the human prostate. Prostate tissue comprises 3 histologically different zones: a) peripheral, b) central, c) transitional(TZ). The TZ normally takes up 5% of the gland and is the only zone in which BPH develops (McNeal and collaborators J. Pathology of benign prostate hyperplasia. Insight into etiology. (Urol Clin North Am. 1990; 17: 477) An increase in the stromal compartment within the TZ tissue, leading to obstruction of urinary outflow, has been described as being the most prominent morphological difference between normal prostatic and BPH tissue (Ishigooka M. et al. Prostate 1996; 29: 77). Most likely, BPH is intrinsically a mesenchymal disease, resulting from a re-awakening of mesenchymal interactions between prostatic stroma and epithelium (Bierhoff E. The Prostate 1997; 31: 234-240).

Studies over recent years have identified intra-prostatic signaling systems that are clearly important for the regulation of stromal cell proliferation and extra-cellular matrix production, the two major elements of prostatic stroma. Central to this is an apparent balance between polypeptide growth factors, including members of fibroblast grow factors (FGF), transforming grow factors (TGF) and connective tissue grow factors (CTGF). In BPH there is a lack of balance in the production of these three grow factors: FGF, TGF and CTGF. Up regulation of FGF generates increased prostatic cell proliferation. TFG-B1 up regulation stimulates collagen synthesis by inducing CTGF in stromal cells. Over expression of CTGF in BPH leads to excessive collagen accumulation, as seen in fibrotic disorders like Crohn's disease, systemic sclerosis or keloids.

There is a good evidence that above elements of this system are over expressed in BPH tissue. Potential modulators of the balance between these factors include senescence, estrogens, androgens, adrenergic signaling and inflammation, as causing BPH. Prostatic inflammation is an extremely common histological finding in patients with symptoms of BPH who apparently have no symptoms of prostatitis (Nickel J. C. et al. BJU International 1999; 84: 976-981) Bacterial presence associated with BPH, has been documented in patients with BPH (Hasner E et al. Acta Chir Scanda. 1962; 2: 1-516) Hochreiter et al. proved a significant correlation between bacterial 16 S rDNA and BPH using a highly sensitive polymerase chain reaction (PCR) assay (Hochreiter W. W J Urol. 2000; 163 (1) : 127-30) These increased levels of bacterial 16 S ribosomal DNA are compatible with the notion that a bacterial inflammatory event initiates the mesenchymal re-awakening. These receptors are found in the cells of different tissues, e.g.: intestinal mucose, prostate, innate immune system, etc.

In the past years important advances were obtained as to the biology of TOLL-like receptors (TLR), which have been shown to play important roles in host cells responses toward bacteria, bacterial products, fungi and viruses. Different bacterial products mediate cellular activation via distinct TLR (Akira S. Toll-like receptor signaling. Nature Review Immunology 2004; 4: 499) Activation of TLR-dependent signaling pathways leads to the activation of immune responses including the expression and release of various pro-inflammatory and chemotaxant cytokines.

Furthermore, bacterial products are capable of promoting fibrosis (Tol v. E.A.F. et al. Am. J. Physiol. 1999; 40: 277) Increased extra-cellular collagen synthesis and concomitant increased TGF-1B and interleukin (IL)-6 expression was found by direct stimulation of mesenchymal cells. Neutralization of TGF-1B, inhibited in vitro collagen gene expression.

Pseudomonas Aeruginosa Hapten

Recently, an in-depth immunological study on BPH has brought to light the relationship between this disease with immunological mechanisms, specially the important intra-prostatic signaling systems and the Toll like-receptors mentioned above, not so well-known so far outside of Immunology. Now perhaps, we can better understand the therapeutic possibility of PAH over BPH (Kuijpers K. A. J. Haptenotherapy for the treatment of benign prostatic hyperplasia. Dept. of Exper. Urol. Niejmegen University and Medical Center. The Netherlands. March 2005)

An hapten is a non-immunogenic part unable of acting by itself. However, when attached to a lipid and protein carrier, it becomes an antigen, causing fever and toxic condition on one hand, but stimulating defense mechanisms on the other. Toxic effects and fever are originated in the lipid fraction. By obtaining in 1955 a lipid free Pseudomonas Aeruginosa Hapten (PAH), (Puebla LC et al. Treatment of allergic diseases with the Pseudomonas Aeruginosa Polysaccharide. IV SC. Sect.; August 1955) found that PAH was not acting toxic but as a desensing immunomodulator. Pseudomonas Aeruginosa (PA) is a Gram-negative bacteria; unlike Gram-positive, have lipopolysaccharides (LPS) embedded in their outer cell wall layer. These LPS are recognized by the TOLL-like receptors of the innate immune system. Eleven members of the TOLL-like receptor have been identified in mammals. TLR 7 and TLR 9 are involved in the recognition of nucleic-acid-like structures. TLR 4 is involved in the recognition of polysaccharides.

The Pseudomonas Aeruginosa Hapten is compounded with three different elements: the polysaccharide itself, remains of nucleic acids (RNA or DNA) and a polypeptide or carrier. All three elements are believed to work as immunomodulator on the TOLL-like recognition system.

Strong expression of TLR's in prostate fibroblasts was found by Konig J. E. et al in BPH tissue. (The Prostate 2004; 58: 121-129) Although more research would be needed regarding that, it seems obvious that TOLL-like receptor depending inflammatory cascades within the prostate tissue are crucial in the development of BPH.

According to the TOLL-like recognition system, PA polysaccharides are capable of blocking the direct stimulation of prostate fibroblasts by TLR 4, hereby inhibiting secretion of pro-inflammatory cytokines. At the same time, PAH is claimed to stimulate leukocyte migration and neutrophilic granulocytes resulting in increased reabsorption of necrotic and infarction focuses, often found in BPH tissue. Bacterial nucleic-acids recognition of TLR 9 is possibly involved in this matter.

Benign Hyperplasia of Prostate is one of the most common diseases affecting men and the number of cases increases year after year (Carter H B, Coffey D S Prostate; 16: 38-49, 1990).

Available treatment at present are: surgery, alpha-blockers or hormonal therapy (Caine M: J Urol 136: 1-4, 1986)

On the other hand, it is known that polysaccharides from some Gram negative bacterial wall are useful in the treatment of skin diseases and allergy.

An injected commercial product known as Poligram made out of bacterial polysaccharides it is used in dermatological treatments such as eczema, keloids, hypertrophic scars and ulcer.

The document of Patent EP 1417969 of HPB S.A. has as main objective to make known an injected liquid composition which includes polysaccharides from Gram negative bacteria, hydro-soluble extract of Thymus and hydro-soluble extract of Prostate. The disclosed way of application is complex and longer, lasting 4 months. Subcutaneous injections of 1 mg are applied daily from day 0 up to 20°; then 0.5 mg. from day 21° till 45°, 6 days in a week (Sunday to rest); then 0.5 mg from day 46° till 66°, 5 days a week; from day 67° till day 90°: 0.5 mg 4 days in a week; and finally, 0.5 mg. 3 days in a week (Monday, Wednesday and Friday) from day 91° up to day 120°.

### SUMMARY OF THE INVENTION

It is an objective of the present invention to ascribe to a solid pharmaceutical composition to treat Hyperplasia Benign of Prostate, in which each unit (1 ml.) of said composition contains at least between 0.5 and 5 mg., of polysaccharide extract of Gram negatives wall bacteria and pharmaceutical acceptable excipients. Said bacteria preferentially Pseudomonas Aeruginosa. Excipient are i.e.: colloidal silicic anhydride, wheat starch, micro crystalline cellulose, lactose or a combination of both. The compound can be prepared as pastilles, capsules, tablets or pills. Preferentially the composition is made as enteric capsules.

Another objective of the present invention is to provide a method of preparation of the said solid composition for the treatment of Hyperplasia Benign of Prostate which includes the following stages:
A) to culture bacteria Gram negative; preferentially Pseudomonas Aeruginosa, in a nutritious agar broth;
B) to extract the lipo-polysaccharides from the bacterial walls and purified it;
C) to separate the lipoid fraction in order to obtain polysaccharides in maximum purity;
D) to dry the solution of polysaccharides and mill it; and to prepare a solid composition which at least includes the polysaccharides extract dried and milled in the previous stage.

It is another objective of the present invention to secure a procedure for the treatment of benign hyperplasia of prostate, in which said method consists in providing to a needed individual a therapeutically effective quantity of a polysaccharide extract from a wall of a Gram negative bacteria. The quantity of the said polysaccharide extract may be between 0.5 mg and 5 mg per day, more preferentially 1.5 mg. per day. Preferentially the administration of the composition of the invention is oral.

### BRIEF DESCRIPTION OF THE DRAWINGS

FIG. 1: It is a graphic showing the patient response to the treatment with the capsules of the invention, known in these figures as ORAL PAH. Parameters evaluation were performed on day Zero, 25° and 70° from the beginning of treatment. They were: Q max; post voided residual urine; digital rectal examination (DRE); prostate volume, Prostate specific antigen (PSA) and the original Boyarsky's IPSS (International Prostate Score System), as described in Sample 5.

FIG. 2: It shows response of patients treated with the injected composition of the PREVIOUS ART. Parameters evaluation were done on days Zero, 45° and 120° of treatment and they were Q max. and post voided residual urine, as commented on Sample 5.

FIG. 3: It shows response of patients treated with the injected composition of previous art. Parameters evaluation done during days zero, 45° and 120° of the trial, and were digital rectal examination (DRE) and the volume of prostate, as detailed on Sample 5.

FIG. 4: Showing patient response treated with the injected composition of previous art. Parameters evaluations were done during days zero, 45° and 120° of the study, and they were: prostate specific antigen (PSA) and IPSS, Boyarsky International Prostate Score System, as detailed on Sample 5.

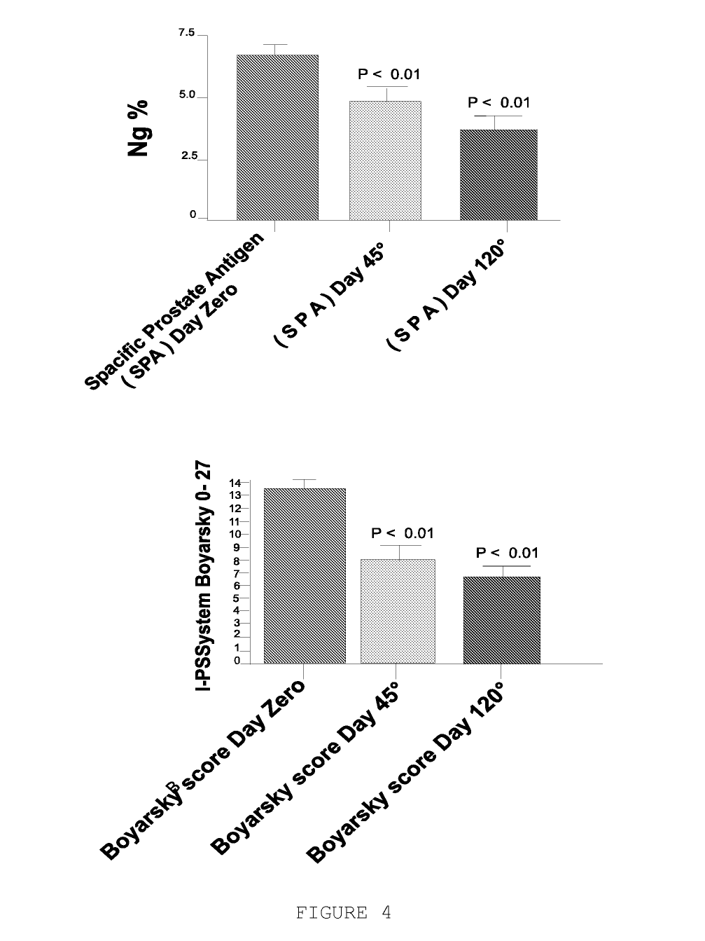

### DETAILED DESCRIPTION OF THE PREFERRED EMBODIMENT

The solid pharmaceutical composition of the present invention exceeds the previous art disclosed as injected compositions because reduces the problem to the administration of one capsule of the invention per day which consist of 1.5 mg. only during 70 days of treatment; so, the oral polysaccharide complex would be absorbed therefore acting in a more efficient way through out the intestinal mucosae, very rich in Toll-like receptors. Moreover it is possible to low the production costs; and finally because we avoid the common repulse to injections.

The solid pharmaceutical composition of the present invention includes an extract which contains polysaccharides of Gram negative bacteria and suitable excipient. The composition may be presented as tablets, capsules, pastilles and pills, being the capsules the preferred composition, prepared with cellulose acetoftalate and poly-ethyl englicoll 6000. Experts in the art know that it is possible to utilize polysaccharides extracted from the cellular wall of any Gram negative bacteria, being insomuch that all the polysaccharides from the cellular wall and the mixtures of both, are available to the present invention.

In the preparation of the composition of the invention is possible to employ any excipient, as long as the formula be resistant to the stomach pH and so, the active principle be liberated into the first intestinal portion.

In the essays performed to patients they were treated with a capsule per day of the composition of the invention, which capsule contained 1.5 mg of the polysaccharide extract from the cellular wall of the Pseudomonas bacteria. The placebo group received capsules containing only the excipient. Capsules for the treated group were prepared according to the disclosed method described in the Samples detailed below. A total of 87 patients were included in the study, 44 were into the group treated with the composition of the invention and 43 patients were in the control group which received the placebo capsule.

We are hereby using the word PAH to define the group treated with the composition of the invention. Results of the treatment are shown in Table 1.

TABLE 1
Clinical comparative response between the groups
treated with the solid composition of the invention and
Placebo

| | PAH | PAH | PAH | Placebo | Placebo | Placebo |
|---|---|---|---|---|---|---|
| | n = 44 | n = 44 | n = 44 | n = 43 | n = 43 | n = 43 |
| | Day 0 | Day 25 | Day 70 | Day 0 | Day 25 | Day 70 |
| AGE | 66 ± 5 | | | | | |
| Q MAX./ml. | 7.7 ± 45 | 10.8 ± 1.4 | 14.7 ± 1.9 | 6.0 ± 1.2 | 8.6 ± 3.6 | 15.7 ± 1.5 |
| RESIDUAL | 191 ± 48 | 90 ± 19.3 | 50. ± 10 | 175 ± 46 | 139 ± 49.5 | 46 ± 16 |
| URINE/ml | | | | | | |
| Digital | 3.8 ± 0.7 | 2.5 ± 0.6 | 1.6 ± 0.4 | 3.7 ± 0.3 | 3.2 ± 0.9 | 1.6 ± 0.2 |
| Rectal | | | | | | |
| Exam (DRE) | | | | | | |
| (0–4) | | | | | | |
| Prostate/ | 69 ± 19 | 63 ± 18 | 55 ± 17 | 72 ± 22 | 77 ± 20.5 | 62 ± 17.7 |
| Volume ml | | | | | | |
| S. Prostate | 7.1 ± 1.8 | 4.5 ± 2.2 | 3.1 ± 1.9 | 7.0 ± 1.1 | 5.9 ± 1.0 | 2.3 ± 0.9 |
| Antigen | | | | | | |
| BOYARSKI | 14.1 ± 0.2 | 7.3 ± 0.6 | 4.6 ± 0.4 | 14.1 ± 0.7 | 12.3 ± 0.5 | 7.6 ± 0.5 |
| SCORE | | | | | | |
| (0–27) | | | | | | |

The patients treated with the solid composition of the invention show clinical signs of improvement to the day 25° of the treatment, being fairly better results in comparison with those of the placebo group. Thereafter, as the placebo group began to be treated after day 25° with the solid composition of the invention, it is observed that towards day 70° the group of control reaches clinical response similar to the PAH group initially treated from Day Zero of the essay.

On the other hand, the trial included comparative clinical studies employing the injected solution of the previous art. So, on Table 2 are described the results obtained with the injected solution in the PAH group of 119 patients in comparison with those 115 patients treated with placebo.

TABLE 2
Clinical results between the groups treated with the
Injected composition of the previous art and the
Placebo

| | PAH | PAH | PAH | Placebo | Placebo | Placebo |
|---|---|---|---|---|---|---|
| | n = 119 | n = 119 | n = 119 | n = 115 | n = 115 | n = 115 |
| | DAY 0 | DAY 45° | DAY 120° | DAY 0 | DAY 45° | DAY 120° |
| AGE | 65 ± 8 | | | | | |
| Q MAX./ | 8.1 ± 56 | 11.5 ± 2.1 | 15.5 ± 2.4 | 7.0 ± 1.4 | 8.3 ± 4.9 | 15.4 ± 2.2 |
| ml. | | | | | | |
| RESIDUAL/ | 188 ± 56 | 89 ± 24.2 | 47. ± 14 | 151 ± 45 | 148 ± 58.1 | 45 ± 15 |
| URINE | | | | | | |
| ml. | | | | | | |
| Digital | 3.1 ± 0.9 | 2.3 ± 0.9 | 1.5 ± 0.6 | 3.4 ± 0.8 | 3.4 ± 0.8 | 1.5 ± 0.5 |
| Rectal | | | | | | |
| Exam (DRE) | | | | | | |
| (0–4) | | | | | | |
| Prostate | 73 ± 24 | 65 ± 21 | 58 ± 18 | 73 ± 24 | 76 ± 23.1 | 63 ± 18.3 |
| Volume/ | | | | | | |
| ml. | | | | | | |
| Sp. Prostate | 6.1 ± 2.9 | 4.9 ± 2.4 | 3.3 ± 2.0 | 6.4 ± 1.0 | 6.1 ± 1.1 | 2.5 ± 1.2 |
| Antigen | | | | | | |
| (SPA) | | | | | | |
| (ng) | | | | | | |
| BOYARSKI | 13.5 ± 0.5 | 7.5 ± 0.5 | 5.5 ± 0.5 | 13.5 ± 0.5 | 14.5 ± 0.5 | 7.5 ± 0.5 |
| SCORE | | | | | | |
| (0–27) | | | | | | |

Here may be observed that only towards Day 45° those patients treated with the injected solution of the previous art, reached similar clinical response to the patients treated with the solid composition of the invention. We conclude therefore that the solid composition of the invention has early positive effects, into a period of time 40% shorter than the injected solution. The solid composition of the invention fulfill the treatment into de 70 days of day zero, whilst the injected solution needs 120 days of treatment. Therefore, the solid composition of the invention allows a shorten treatment and because is orally administrated is better accepted by the patient. Oral administration results to be an easily administered therapeutic way, particularly so for extended and ambulatory treatments.

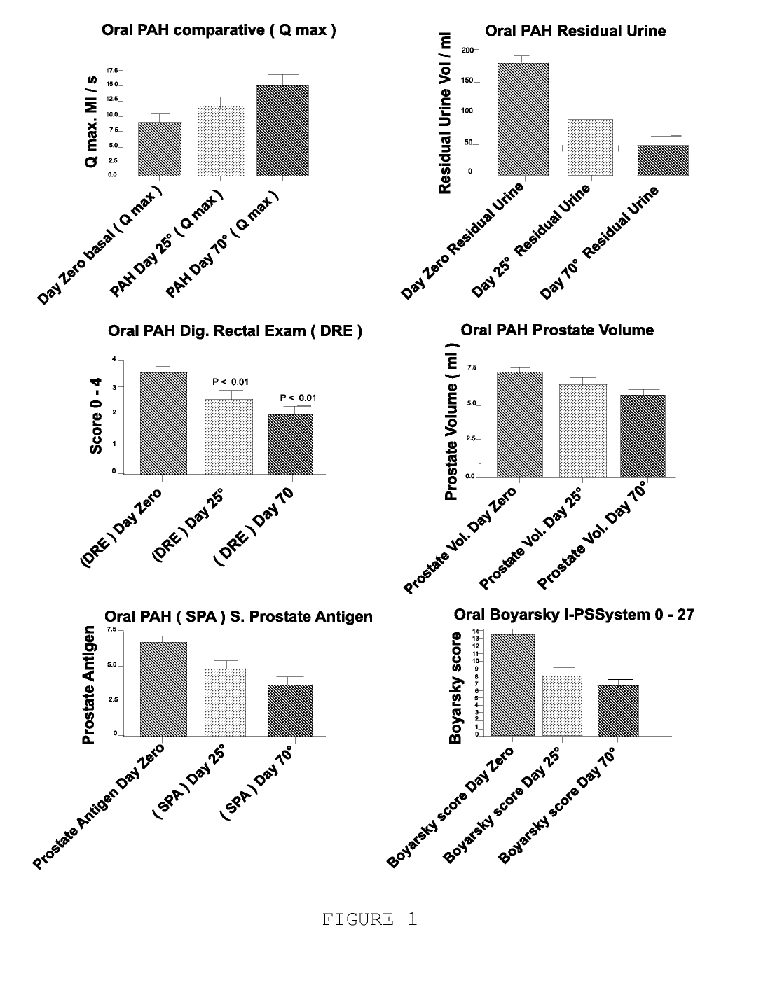

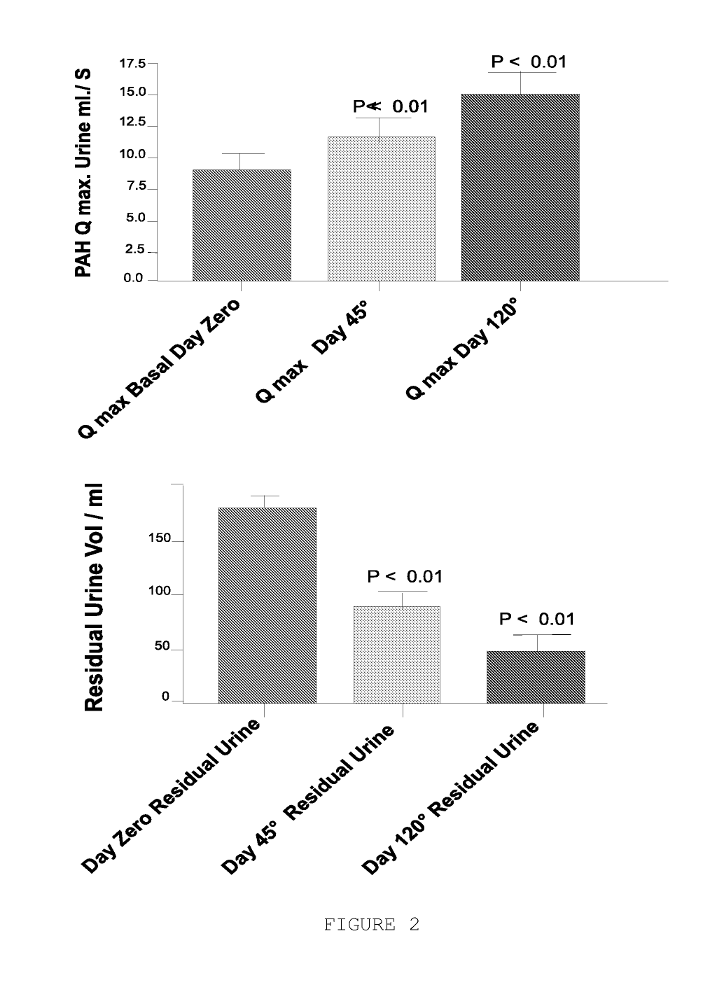

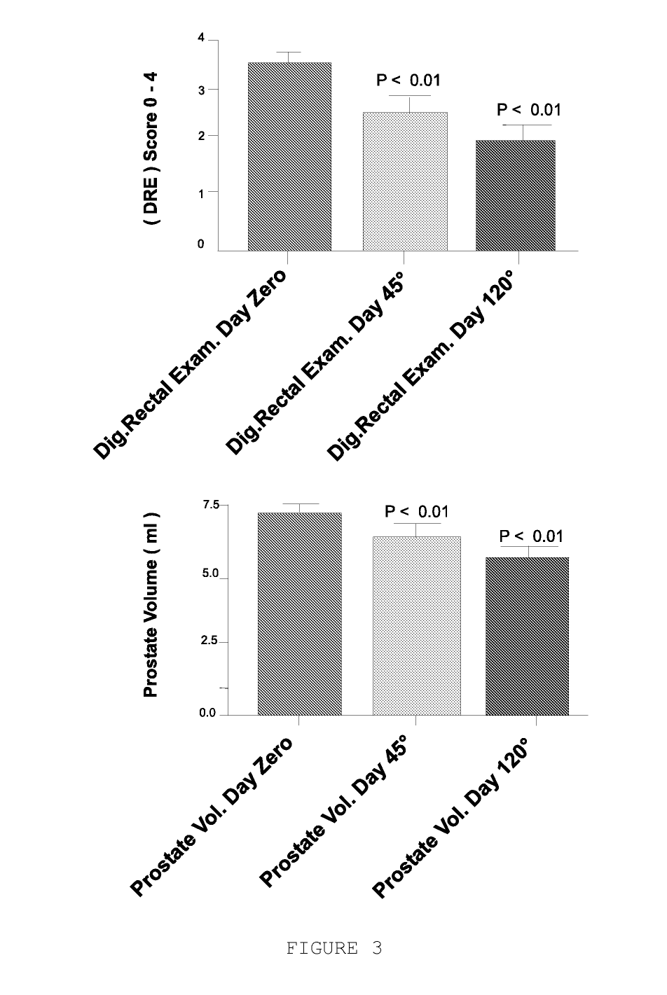

From the comparative analysis (see FIGS. 1, 2, 3 y 4) on pages may be observed that the patients administered with the solid composition of the invention have a positive response to treatment into the 70 days of initiation, in all the evaluated parameters.

This invention is better explained according to the following Samples, which should not be taken just as a simple limitation to it. On the contrary, must be clearly understood that it could be appealed to another performing, modification and equivalences of the same. And after reading through the present Description of the Invention the experts in the matter may be enlightened without driving away from the spirit of the present invention and/or the scope of the annexed claims.

### SAMPLES

Sample 1: Culture of Pseudomonas Bacteria.

Pseudomonas bacteria are sowed into Erlenmeyer jar containing a nutritious cultured broth, under sterility conditions. Growing of bacteria is allowed up to a logarithmic phase. Bacteria are centrifuged and kept frozen till its utilization.

For example, a concentrated cells suspension are sowed in a one liter capacity Erlenmeyer jar containing 250 cc of a liquid cultured medium.

As a common procedure, to obtain the bacterial growing curve, the count of bacteria versus time shall be put into graphics; for that purpose the commercial cultured medium must be prepared according to indications and five Erlenmeyer jars are filled with 250 cc each one. An sterilization procedure follows and when room-temperature is reached the cells stock are seeded and placed in agitator. Between a one hour period of time, measured exactly and on bacteriological condition, 1 mg. of the culture is extracted then to proceed to the bacterial count. The bacterial counting is done thru the culture in a Petri plate, counting the number of developed colonies using nutritious Agar cultured medium, prepared as indicated. If it is necessary dilutions can be added.

Twice successively, 1 mg. of the culture is extracted from each Erlenmeyer jar to proceed with the bacterial counting.

The bacterial growing curves is done with ten results, taking as the optimum culture time the moment the curve reaches the plateau-stage.

Once the optimum culture time is achieved, the bacterial mass production is started. For that purpose an specific number of Erlenmeyer jars are seeded in strict bacteriological conditions under a laminar flux bell. Then we proceed to weigh.

Bacterial are kept under minus 20° centigrade until its final use.

It is understood that in all cases the corresponding bacteriological control of the stock will performed through specific biochemical tests in order to ensure they are bacteria free.

Sample 2: Procedure for the Extraction of Lipo Polysaccharides of Pseudomonas.

Bacteria are cultured in nutritious agar during 24 hours. Sodium chloride 0.15 M, is added to the culture, turned into suspension and centrifuged during 15 minutes to 5,000 rpm. Three washings with acetone are performed and the resulting microbial paste is thinly spread and put to dry. The bacterial dried paste is grinded in a ball-mill and the resulting powder is put through a sieve.

10 gr. of the previously obtained powder is put on suspension in 175 ml of water to 65-68° Centigrade; an equal volume of phenol at 90% previously heated at same temperature is added, then vigorously agitated. The mixture is kept to 65° C. during 10 to 15 minutes; placed in an iced-bath to cold at 10° C. and centrifuged at 3,000 rpm during 45 minutes. It is obtained an aqueous layer, another phenolic and a third one of unsolvable leftovers. The aqueous layer is separated and kept apart. The phenol layer and the unsolvable leftover are extracted again with 175 ml of hot water, as previously explained. Aqueous extracts are mixed and dialyzed with distilled water in order to eliminate rests of phenol and low molecular weight substances of bacterial origin. Under lower pressure, the opalescent solution contained into the polysaccharide and the ribonucleic acid, is then concentrated to a temperature of 35-40° C. until a volume of 50 ml is reached; then is centrifuged in order to discard unsolvable remnants. The floating material is to be lyophilized.

The lyophilized material is dissolved into distilled water and in a proportion of 150 ml. for each gr. of remnant we add 15 ml of cetyl tri methyl ammonium bromide (Cetavión), 2% in water. The mixture is to be agitated at room-temperature during 15 minutes and then centrifuged 20 minutes at 3,000 rpm in order to discard the precipitated RNA. Floating material is to be lyophilized and then be dissolved again into 50-60 ml of sodium chloride 0.5 M. Finally, lipo-polysaccharides are precipitated by adding of five ethanol volume. Placed at 4° C. during 2 hours, it is centrifuged and dissolved again into a convenient volume of distilled water.

The lipo-polysaccharide so obtained represents approximately 2% of the dry weight of processed bacteria.

Sample 3: Procedure to Obtain Polysaccharides.

From the lipo-polysaccharide obtained in the previous phase, a (10 mg/ml) solution in acetic acid is prepared at 1%; it is heated at 100° C. during 30 minutes in order to produce hydrolysis and then centrifuged at 500 g to separate the unsolvable lipid A. The floating material is dialyzed with distilled water at 4° C. during 48 hours and finally the obtained product is lyophilized. As a result of this process the obtained product is a polysaccharide complex compounded by three elements: a) the polysaccharide itself; b) remnants of nucleic acid (DNA & RNA); and c) amino acids & low molecular weight polypeptides. Through the TLR (Toll-like- Receptors), polysaccharides and particularly the nucleic acids, would work through immune-modulators mechanisms, according to description in the precedent bibliography.

Sample 4: Preparation of the Solid Pharmaceutical Composition of the Invention.

A concentrated solution of 5 mg./ml from the polysaccaride is prepared. Solution is put to dry with silicic anhydride, then the following excipients are added: Colloidal silicic anhydride, wheat starch, micro crystalline cellulose and lactose. Enteric coverage is done with cellulose acetophtalate and Poly ethilenglycol 6,000. Mixture is homogenized and then encapsulated. The procedure ends with the enteric covering.

Sample 5: Clinical Research with the Pharmaceutical Composition of the Invention in Patients with Benign Prostate Hyperplasia (BPH).

One hundred twenty six patients with BPH and urine problems participated at the clinical essay

Exclusion criteria: prostate cancer, urinary stasis leading to renal failure; Detrusor instability or incompensation; cerebro-vascular illness; severe neurological or psychiatric disorders; urethral post-traumatic stricture; large diverticulum of the bladder; bladder calculi; neurological bladder; previous prostate surgery; chronic prostatitis or other disorder post invasive procedure.

Inclusion criteria: all patients with BPH symptoms were evaluated according to the (I-PSS) International Prostate Score System Boyarsky (0-27 scoring); urodynamic testing (Urobite 1000, Promedon 1997); post micturition residual volume( assessed by Ultrasound; Specific prostate antigen (SPA). In order to rule out prostate cancer, in all cases with SPA>than 4 ug/ml a biopsy was performed.

Prostate volume was monitored by trans-rectal ultrasound and a digital-rectal examination (DRE) to evaluate consistency and hardness of the prostate gland. This parameter was divided into 4 degrees: 1^{st}. degree=very soft; 2^{nd}. degree=soft; 3^{rd}. degree=hard and 4^{th} degree=very hard. All participants with 4^{th} degree consistency were performed prostate biopsy

Randomization: Patients were divided in two groups: the active group received the solid composition of the invention and the control group treated with placebo. After a detailed explanation of the research program to the candidates, the informed consent was signed.

Eighty seven candidates were finally accepted in the double-blind, randomized and placebo controlled study. The active group (44 patients) were treated with the composition of the invention without interruption during 70 days (until day 70°) in a single dose of 1.5 mg per day. Control group (43 patients)received instead placebo capsules, from day Zero to day 25°. Thereafter, (from day 26° ahead) began the treatment with capsules 1.5 mg, daily dose of the composition of the invention until a full period of 70 days was completed. Meaning that both groups (active and control groups as well) were treated during 70 days with the composition of the present invention with a daily capsule of 1.5 mg each. Thereafter all tests were concluded. All laboratory proofs, Boyarsky I-PSS and additional tests—done on Day Zero—were repeated on day 25° and day 70° in every patient of both groups.

It has been specially described and exemplified the nature of the present invention, as well as the way in which the invention can be put into practice, it is hereby claimed the ownership and the exclusive legal rights:

1. A solid pharmaceutical composition for the treatment of Benign Prostate Hyperplasia duly characterized because each Unit contains at least between 0.5 and 5 mg. of an extract of polysaccharide of the Gram negative bacteria wall and also pharmaceutically acceptable excipients.

2. The composition, according to claimed in 1, it is characterized because excipients are a selection of the group compounded by colloidal silicic anhydride, wheat starch, micro-crystalline cellulose, lactose and a combination of both.

3. According to claimed in 1, the composition is duly characterized because the said solid composition is into the selected group compounded by pastilles, capsules, tablets and pills.

4. According to claimed in 1, the composition is duly characterized because the extract includes moreover nucleic acids and polypeptides.

5. According to claimed in 1, the composition is duly characterized because the bacteria is Pseudomonas Aeruginosa.

6. A procedure to prepare a solid composition for the treatment of the hyperplasia benign of prostate, characterized because includes the phases of:
A) to culture in a nutritive agar medium Gram negative bacteria;
B) to extract lipo-polysaccharides from the said bacteria wall and purified it;
C) to separate the lipid fraction in order to obtain the purified polysaccharides;
D) to dry the obtained polysaccharides solution, then mill it; and
E) to prepare a solid composition which at least includes the polysaccharide extract dried and milled in the previous phase.

7. According to claimed in 6, the procedure is characterized because the Gram negative bacteria is Pseudomonas Aeruginosa.

8. According to claimed in 6, the procedure is characterized because the solid composition prepared in phase e) is a selected composition of the group integrated by tablets, capsules, pastilles and pills.

9. According to claimed in 6, the procedure is characterized because in phase e) are added besides, pharmaceutically acceptable excipients.

10. A method for the treatment of hyperplasia benign of prostate, duly characterized because it includes the administration to a needed individual of a quantity therapeutically effective of an extract of a Gram negative bacteria wall polysaccharide.

11. According to claimed in 10, the method is characterized because the administration consist of a quantity between 0.5 and 5 mg per day.

12. The method according to claimed in 11, is characterized because 1.5 mg is administered per day.

13. The method according to claimed in 10, is characterized because the extract of polysaccharides is in a solid way.

14. The method according to claimed in 13, is characterized because the solid manner is selected from the group integrated by tablets, capsules, pastilles and pills.

15. The method according to claimed in 14, is characterized because the solid form is an enteric coat capsule.

16. The method according to claimed in 10, is characterized because the Gram negative bacteria is Pseudomonas Aeruginosa.

17. The method according to claimed in 10, is characterized because the polysaccharide extract includes besides nucleic acids, amino acids and polypeptides.

18. The method according to claimed in 10, is characterized because it is administered by oral way.
